(19) United States
(12) Patent Application Publication
(10) Pub. No.: US 2007/0280406 A1
(43) Pub. Date: Dec. 6, 2007

(54) ORIENTATION IDENTIFICATION

(76) Inventor: David Geliebter, Palm Beach Gardens, FL (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/757,994

(22) Filed: Jun. 4, 2007

Related U.S. Application Data
(60) Provisional application No. 60/803,920, filed on Jun. 5, 2006.

(51) Int. Cl. A61K 49/04; G01T 1/10

(52) U.S. Cl. 378/4; 424/9.4

(57) ABSTRACT

A pair of anatomic orientation stickers may include a left-hand sticker comprising a radiopaque material forming a figure “L” and an adhesive backing attached thereto, and a right-hand sticker comprising a radiopaque material forming a figure “R” and an adhesive backing attached thereto. The stickers may be sufficiently flexible and biocompatible, and the adhesive backings are sufficiently adhesive, to permit removable application on a curved skin surface.

### CROSS-REFERENCE TO RELATED APPLICATION

This application claims the benefit of U.S. Provisional Application No. 60/803,920, filed Jun. 5, 2006 under attorney docket No. CCK-001.60, and which is hereby incorporated herein by reference.

### SUMMARY

Devices, systems, and methods are described for, among other things, identifying the orientation of a subject undergoing an imaging procedure.

A pair of anatomic orientation stickers may include a left-hand sticker comprising a radiopaque material forming a figure “L” and an adhesive backing attached thereto, and a right-hand sticker comprising a radiopaque material forming a figure “R” and an adhesive backing attached thereto. The stickers may be sufficiently flexible and biocompatible, and the adhesive backings are sufficiently adhesive, to permit removable application on a curved skin surface.

A method of marking an anatomic orientation on a subject may include affixing the left-hand sticker of the sticker pair to skin overlying a left-hand anatomic structure of the subject and/or affixing the right-hand sticker of the sticker pair to skin overlying a right-hand anatomic structure of the subject.

### BRIEF DESCRIPTION OF DRAWINGS

FIG. 1 depicts an exemplary embodiment of a pair of anatomic orientation stickers.

FIG. 2 depicts an exemplary embodiment of a pair of anatomic orientation stickers releasably adhered to a sheet.

FIG. 3 depicts another exemplary embodiment of a pair of anatomic orientation stickers.

FIG. 4 depicts an exemplary embodiment of an anatomic orientation sticker including a nonadhesive flag.

FIG. 5 depicts an exemplary embodiment of an anatomic orientation sticker disposed in a dispenser.

FIG. 6 depicts an exemplary embodiment of a pair of anatomic orientation stickers having a protective coating.

FIG. 7 depicts a simulated x-ray of a right knee with an “R” orientation sticker placed on the right shin.

FIG. 8 depicts the simulated x-ray of FIG. 7 reversed.

FIG. 9 depicts a right-hand glove including a radiopaque figure “R”.

### DETAILED DESCRIPTION

Orientation errors when viewing medical images may result in subsequent medical treatment errors. For example, if an x-ray is read in a flipped-over orientation, the healthcare provider reading the image may mistake left-sided features for right-sided features or vice-versa. Such mistakes may lead to serious consequences, such as surgery or other medical interventions being carried out on the wrong body parts.

One or more landmarks may be provided on a medical image to help ensure that the image is read in the correct orientation. The landmark should be made in whole or in part of a material that will render the landmark visible on a given medical image. For example, if the image is an x-ray (or x-ray-based imaging such as computed tomography, angiography, and the like), then the landmark should include a radioopaque ingredient. For a visible-light image, the landmark should include an ingredient that absorbs light, reflects light, emits light, or perturbs light interacting with it.

In one embodiment, landmarks may be stickers. The stickers may be shaped, or may include shapes, that provide unambiguous orientation indications. For example, stickers may be shaped as or may include shapes such as “R” and “L” to stand for right and left, respectively (other letter combinations suitable for other languages are contemplated, such as “D” and “G” in French, etc.). Instead of letters, asymmetric symbols may be used (such as “LEFT”, “RIGHT”, “THIS SIDE UP” or “READ THIS WAY”). If two landmarks are used on an image, they may be so chosen as not to be horizontally and/or vertically symmetrical with one another. The stickers may be placed on a subject's skin before a medical image is obtained. The stickers' shapes will be captured as part of the imaging procedure and will appear on the medical image. The asymmetric landmark shape will then help a subsequent reader ensure that the image is correctly oriented.

Instead of stickers, landmarks may be provided as ink. The ink may be disposed in a pad and is applied to the subject using a stamp, or the ink may be disposed in a pen or other marker and is applied to the subject by drawing on the subject with the pen or marker.

Landmarks may be designed for single use. For example, stickers may be provided in quantity on a sheet. When a subject is to be imaged, the imaging technician peels one or more appropriate stickers from the sheet and applies it/them to the subject. After the imaging procedure, the sticker(s) may be peeled off the subject and discarded. A stamp-and-pad or a pen/marker may be provided with a small quantity of ink appropriate for one or two applications. Alternatively, the landmarks may be designed for re-use; a stamp-and-pad or pen/marker may be re-used repeatedly and/or may be sterilizable.

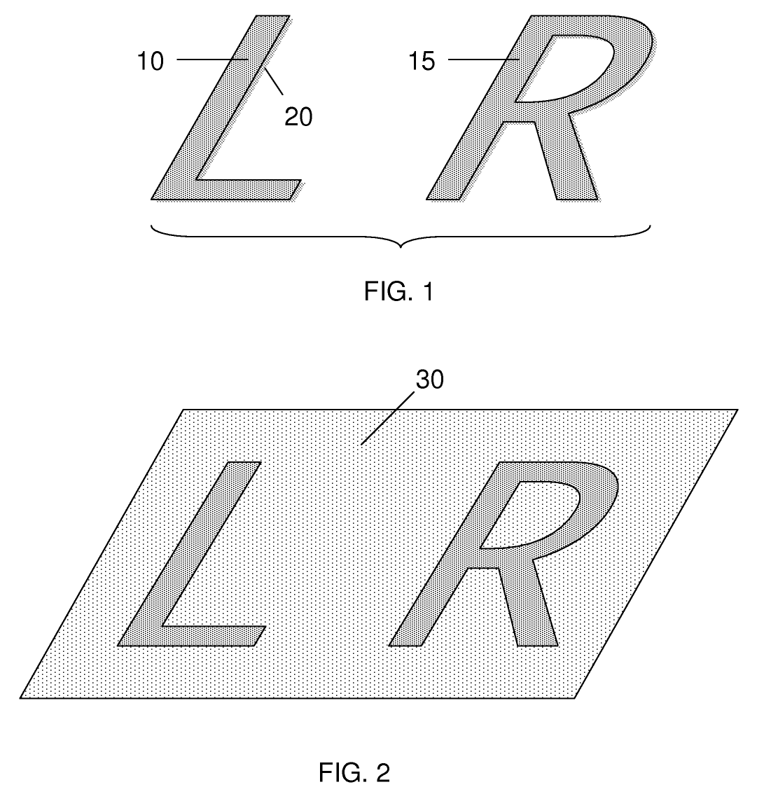

FIG. 1 shows an exemplary embodiment of a pair of stickers, the left-hand sticker forming a figure “L” and the right-hand sticker forming a figure “R”. Each sticker includes a radiopaque material 10, 15 and an adhesive backing 20 attached to it. (The drawings include some space between the layers for clarity; in typical use, the layers would be in full contact with one another.) The stickers are so biocompatible as to permit application on a subject's skin. They are also sufficiently flexible to allow them to be applied to a curved skin surface; i.e., the stickers can flex to follow the skin contour. The adhesive backing includes an adhesive that is sticky enough to adhere the sticker to the skin. The adhesive may be sticky enough to permit temporary adhesion but not so sticky as to prevent removal without injury and/or discomfort to the subject. An example of a suitable adhesive is an adhesive that is used on flexible plastic or fabric bandages.

A wide variety of radiopaque materials may be used, such as barium, bismuth, tantalum, tungsten, lead, aluminum, compounds of those materials, and mixtures including those materials. The radiopaque material may be provided in a wide variety of formats, such as a thin foil or sheet or in particle form. Particles of radiopaque material may be dispersed in a carrier material, such as a liquid carrier, to facilitate application of the radiopaque material to a substrate by, e.g., spraying.

The adhesive backing may take a wide variety of forms. For example, the adhesive backing may be a sheet onto one side of which is applied an adhesive material. The sheet may be plastic sheet. The adhesive backing is typically made of radiolucent material to avoid obscuring radiogaphs of the underlying anatomy.

The adhesive backing and the radiopaque material should be positioned relative to one another so that the figure formed from the radiopaque material reads correctly when the adhesive is applied to the skin. For example, the adhesive backing should be underneath a figure “L” when the “L” is in the normal reading configuration; the adhesive should be above the figure “L” when the “L” is reversed, as in “ [symbol] ”.

FIG. 2 shows a pair of stickers disposed on a sheet 30. The sheet protects the adhesive backings from exposure prior to use. The stickers may be peeled from the sheet for use. The stickers may be so releasably adhered to a sheet as to leave the adhesive backings intact with the respective radiopaque materials when the stickers are peeled from the sheet.

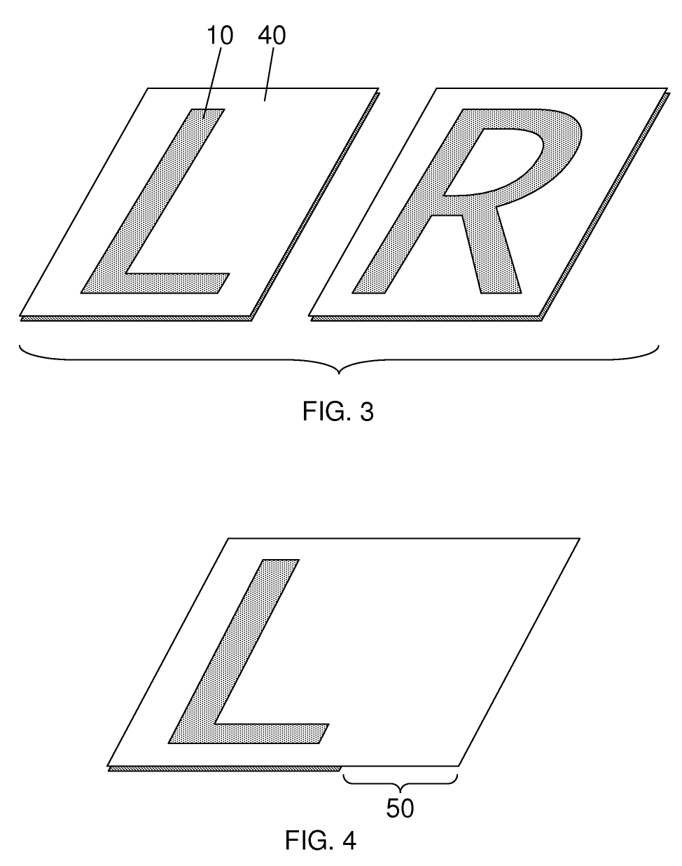

The stickers of the embodiments shown in FIGS. 1 and 2 take the shape of their respective figures (i.e., the sticker bearing a figure “L” is itself shaped like an “L”). Alternatively, the sticker may include non-radiopaque regions, such as non-radiopaque region 40 shown in FIG. 3. Non-radiopaque regions that complement the radiopaque material may permit the creation of a sticker that has a regular shape, such as rectangular or circular. The may also permit the creation of a sticker that is significantly larger than the figure defined by the radiopaque material; the large size provides a large surface area of contact between the sticker and the subject's skin, which improves adhesion without obscuring more of the patient's tissue from medical imaging than is necessary for orientation identification.

FIG. 4 depicts a sticker that includes a non-adhesive flag 50. The adhesive backing may include a region that lacks adhesive. The non-adhesive flag provides a convenient place for a user to grip and position the sticker without having the sticker adhere to the user's finger(s).

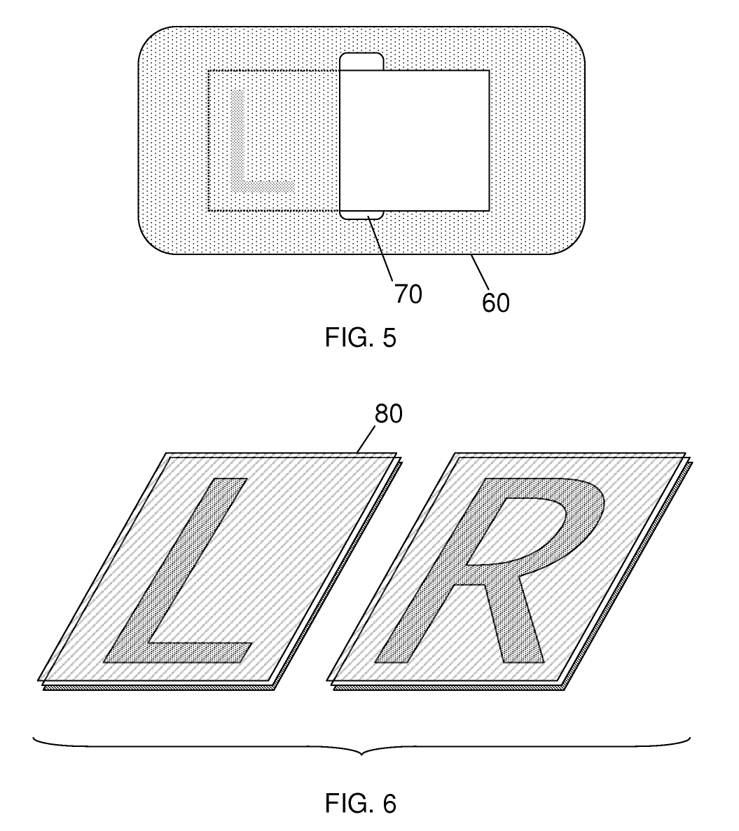

FIG. 5 shows a sticker disposed in a dispenser 60. The dispenser may define an aperture 70 through which the sticker can be dispensed. The sticker may include a non-adhesive flag. A stack of stickers may disposed in the dispenser with the adhesive portion of one sticker releasably adhered to the flag of the next sticker, such as in 3M® POST-IT® flag dispensers. When the first sticker is pulled through the aperture, it drags along the flag of the next sticker through the aperture and simultaneously pulls away from that flag, thereby freeing the flag of the next sticker for dispensing. A dispenser for “L” stickers and a dispenser for “R” stickers may be provided. They may be attached to one another or a common substrate to provide a convenient source of stickers.

FIG. 6 depicts stickers having a protective coating or layer 80 overlying the respective radiopaque material. A protective coating or layer may be used to encapsulate bio-incompatible materials, such as certain radiopaque materials, or to protect a friable or otherwise fragile radiopaque material (such as a foil) from damage. The protective coating or layer may itself be biocompatible. The protective coating or layer may also strengthen the sticker to make it more durable.

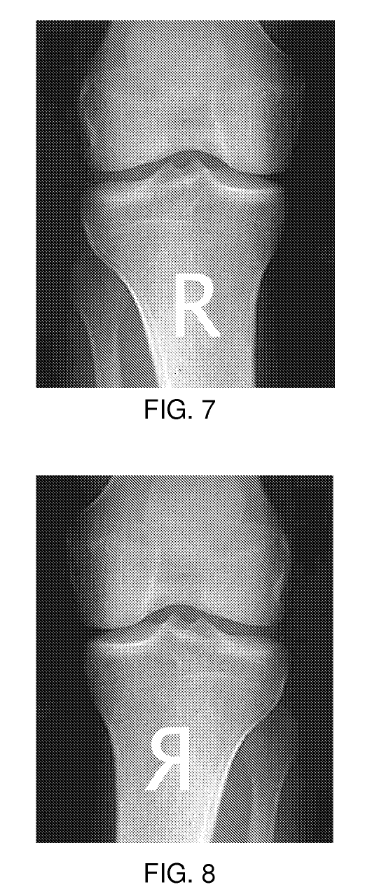

FIG. 7 depicts a simulated x-ray of a knee of a subject who has placed an “R” sticker on the shin. The figure “R” shows up clearly in the x-ray because it is made from radiopaque material. The asymmetric “R” unmistakably indicates that the radiograph is of the right knee. FIG. 8 shows the simulated x-ray of FIG. 7 reversed; the figure “ [symbol] ” clearly indicates the reversal.

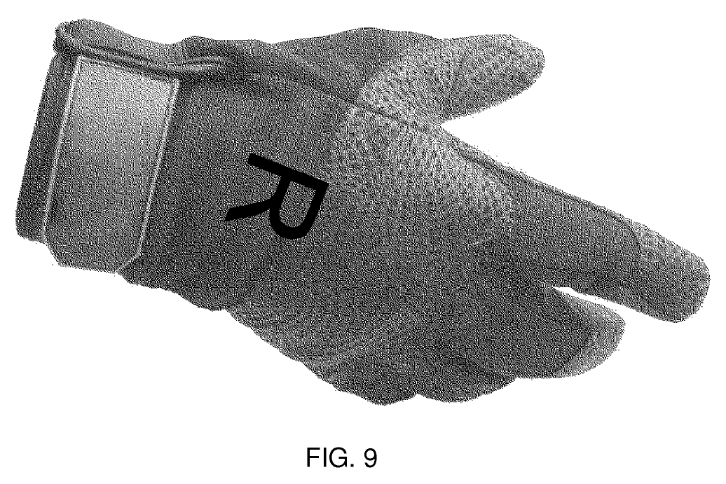

A glove, shoe, or other garment that is specifically shaped or designated to fit on a subject a certain way may include radiopaque material in the form of a figure that can be used to identify anatomic orientation. For example, FIG. 9 depicts a glove that includes a figure “R” formed from radiopaque material. The remainder of the glove may be radiolucent to avoid obscuring a radiograph of the underlying anatomy. Because the glove is specifically shaped for the right hand, the subject is unlikely to place the glove on the left hand. The garment need not be specifically shaped or designated to fit a certain way; for example, the garment can take the form of a piece of material that is wrapped around the anatomy of interest; however, care should be taken to ensure that the radiopaque marker correctly describes the anatomy. The radiopaque marker may thus be formed in such a way that it is visible to the user, such as by incorporating a visible dye.

In use, a left-hand sticker may be applied to skin overlying a left anatomic part, and/or a right-hand sticker may be applied to skin overlying a right anatomic part. A user may apply the sticker(s) to a subject, or the subject may be instructed to apply the stickers him- or herself. Radiographic image(s) of the anatomic part(s) may then be obtained that include the sticker(s). The sticker(s) may be removed after imaging is finished. Radiographic images include images obtained using x-rays, for example, roentgenograms and computed tomography (CT) images.

1. A pair of anatomic orientation stickers, comprising:
a left-hand sticker comprising a radiopaque material forming a figure “L” and an adhesive backing attached thereto; and
a right-hand sticker comprising a radiopaque material forming a figure “R” and an adhesive backing attached thereto;
wherein the stickers are sufficiently flexible and biocompatible, and the adhesive backings are sufficiently adhesive, to permit removable application on a curved skin surface.

2. The stickers of claim 1, wherein the left-hand sticker and the right-hand sticker are both so releasably adhered to a sheet as to leave the adhesive backings intact with the respective radiopaque materials when the stickers are peeled from the sheet.

3. The stickers of claim 1, wherein the adhesive backings of the stickers each further comprise a nonadhesive flag.

4. The stickers of claim 1, wherein the left-hand sticker comprises radiopaque material forming “LEFT” and the right-hand sticker comprises radiopaque material forming “RIGHT”.

5. The stickers of claim 1, wherein the left-hand sticker is disposed in a left-hand dispenser with other left-hand stickers, and the right-hand sticker is disposed in a right-hand dispenser with other right-hand stickers.

6. The stickers of claim 5, wherein:
the adhesive backings of the stickers each further comprise a nonadhesive flag;
the left-hand stickers in the left-hand dispenser are so releasably adhered to one another as to cause the nonadhesive flag of a left-hand sticker to protrude from the dispenser when a prior left-hand sticker is removed from the dispenser; and
the right-hand stickers in the right-hand dispenser are so releasably adhered to one another as to cause the nonadhesive flag of a succeeding right-hand sticker to protrude from the dispenser when a prior right-hand sticker is removed from the dispenser.

7. The stickers of claim 1, wherein the adhesive backings each comprise a plastic film coated with adhesive on one side, and the radioopaque materials each comprise a radiopaque foil fixed to the respective plastic film.

8. The stickers of claim 1, wherein the adhesive backings of the stickers each comprise plastic.

9. The stickers of claim 1, wherein the radiopaque materials comprise at least one of barium, bismuth, tantalum, tungsten, lead, and aluminum.

10. The stickers of claim 1, each further comprising a protective coating or layer overlying the respective radiopaque material.

11. The stickers of claim 1, wherein the radiopaque materials comprise radiopaque particles dispersed in a carrier material.

12. A method of marking an anatomic orientation on a subject, the method comprising, in any order:
(a) affixing the left-hand sticker of the sticker pair of claim 1 to skin overlying a left-hand anatomic structure of the subject; or
(b) affixing the right-hand sticker of the sticker pair of claim 1 to skin overlying a right-hand anatomic structure of the subject.

13. The method of claim 12, wherein step (a) is performed.

14. The method of claim 12, wherein step (b) is performed.

15. The method of claim 14, wherein step (a) is performed.

16. The method of claim 12, further comprising (c) obtaining a radiographic image of the subject, including at least one of the left-hand sticker and the right-hand sticker, after affixing.

17. The method of claim 16, further comprising (d) removing any affixed stickers after obtaining the radiographic image.

18. The method of claim 16, wherein the radiographic image comprises an X-ray image.

19. The method of claim 16, wherein the radiographic image comprises a computed tomography (CT) image.

20. The method of claim 12, further comprising directing the subject to affix one or both stickers him- or herself.

21. An anatomic orientation sticker, comprising:
a radiopaque material forming a figure indicative of a left or right anatomic orientation; and
an adhesive backing attached thereto;
wherein the sticker is sufficiently flexible and biocompatible, and the adhesive backing is sufficiently adhesive, to permit removable application on a curved skin surface.

22. A method of marking an anatomic orientation on a subject, the method comprising affixing the anatomic orientation sticker of claim 1 to skin overlying either (a) a left-hand anatomic structure of the subject if the figure indicates a left anatomic orientation; or (b) a right-hand anatomic structure of the subject if the figure indicates a right anatomic orientation.
